(19) United States
(12) Patent Application Publication
(10) Pub. No.: US 2011/0045050 A1
(43) Pub. Date: Feb. 24, 2011

(54) NANOEMULSION FORMULATIONS FOR DIRECT DELIVERY

(75) Inventors: Tamer A. Elbayoumi, Peoria, AZ (US); Fonghsu Kuo, Nashua, NH (US); Paul Martakos, Pelham, NH (US); Keith M. Faucher, Milford, NH (US)

Correspondence Address: MORSE, BARNES-BROWN & PENDLETON, P.C.;ATTN: IP MANAGER, RESERVOIR PLACE, 1601 TRAPELO ROAD, SUITE 205, WALTHAM, MA, 02451, US

(73) Assignee: ATRIUM MEDICAL CORPORATION, Hudson, NH (US)

(21) Appl. No.: 12/546,312

(22) Filed: Aug. 24, 2009

(51) Int. Cl. A61K 38/12; A61K 31/7048; A61K 31/436; A61F 2/00; A61P 43/00

(52) U.S. Cl. 424/423; 514/31; 514/291; 977/788; 514/20.5; 514/9.8

(57) ABSTRACT

A nanoemulsion and corresponding methods of making and using systems of single or blended high HLB value surfactant(s) for the emulsification of single or blended oils and vitamin E components in an aqueous phase are provided. The resulting nanoemulsions and methods of administering the nanoemulsions, including a corresponding kit, provide delivery of the nanoemulsions to a patient.

### FIELD OF INVENTION

The present invention relates to the administration of poorly water-soluble emulsified therapeutic agents using microporous devices for controlled local intraluminal delivery.

### BACKGROUND

Poorly water soluble drugs or therapeutic agents are sometimes emulsified in oil-in-water or water-in-oil emulsions to improve solubility and bioavailability of the therapeutic agent. Conventional emulsions have droplet sizes larger than 1 μm. Not only are these droplet sizes susceptible to gravity forces, which causes the emulsion to be thermodynamically unstable (i.e., susceptible to sedimentation or creaming), the size of the droplets impedes the flow rate of the formulation through micro-pores typically found on the balloon portion of certain medical or surgical devices, such as catheters.

Nanoemulsions due to their small droplet size, are more thermodynamically stable, are less susceptible to sedimentation or creaming, and may even appear transparent. Nanoemulsions, in contrast to conventional emulsions, are metastable and can be diluted with water without changing the droplet size distribution. While nanoemulsions are preferable in comparison to conventional emulsions, they require high energy input and or high surfactant (emulsifier) content to obtain the sub-micron droplet sizes and maintain the stability of the emulsion. The preparation of nanoemulsions is generally achieved by either high-shear stirring, high-pressure homogenizers, or ultrasound generators. The smaller the droplet size, the more energy and/or surfactant is required. The higher energy requirements make this preparation route unfavorable for industrial applications. The higher surfactant content makes the resulting emulsion toxic in local delivery scenarios, such as when the emulsion formulation is to be delivered through a micro-porous balloon catheter.

Catheters are utilized in a wide range of medical and surgical procedures to dilate, obstruct or restore patency to body vessels or organs. Such catheters typically have a balloon portion that are either perforated or consists of materials having micro-pores. Therapeutic agents may be delivered via the porous balloon portion to a target site proximate to the balloon portion. However, the flow rate and the uniform delivery of such therapeutic agents through the porous walls of the balloon are difficult to control.

### SUMMARY

There remains a need in the art for a nanoemulsion formulation, of a poorly water-soluble therapeutic agent, that can be prepared using less energy and less surfactant. This nanoemulsion must have droplet sizes small enough to traverse the micro-porous balloon portion of a balloon catheter or other irrigation devices having a porous portion, such that the flow rate of the nanoemulsion formulation and the therapeutic agent can be easily controlled and delivered locally in a targeted manner. It has been surprisingly found that nanoemulsions prepared according to the methods provided herein enable the low-energy, low-surfactant preparation of nanoemulsions that can be delivered via a micro-porous irrigation system to a target site such that the therapeutic agent within the nanoemulsion is effectively administered to the target site.

To solve the above mentioned drawbacks and obstacles, the following novel and non-obvious methods and compositions are provided. The present invention provides methods of producing nanoemulsion pharmaceutical compositions suitable for delivery via medical or surgical devices having a micro-porous portion to a body cavity of a subject. The nanoemulsions of the present invention are prepared by a modified version of a phase inversion temperature (PIT) method, and the nanoemulsions of the present invention employ significantly less surfactant than conventional nanoemulsion formulations due to the novel method of manufacture. As part of the method, a heated vortex method is used to load sparingly water-soluble pharmaceutical agents into an oil phase before incorporation into a final nanoemulsion formulation having significantly improved final concentrations of the therapeutic agent, such that the calculated recovery of the therapeutic agent in the final nanoemulsion formulation is over 90%. The nanoemulsions are prepared by a modified PIT method, such that the particle (i.e., droplet) size is reduced to about 100 nm in comparison to conventional 200-400 nm emulsions. This allows for successful sterilization of the produced pharmaceutical agent nanoemulsion formulations through sterile filters. The following methods and compositions provide exemplary embodiments of the invention.

In one embodiment, the present invention provides a pre-concentrate composition having an oil phase, a therapeutic agent, and an emulsifier component, wherein the ratio of the emulsifier component to the oil phase is about 1:1 or less. In other embodiments, the ratio of the emulsifier component to the oil phase is about 0.4:1 or less or 0.3:1 or less in further embodiments. In certain additional embodiments, the oil phase of the pre-concentrate includes one or more fatty acid oils. The fatty acid oil includes polyunsaturated fatty acids. In other embodiments, the fatty acid oil includes a medium chain triglyceride (MCT). In certain other embodiments, the polyunsaturated fatty acid is an omega-3 fatty acid. In yet further embodiments, the polyunsaturated fatty acid includes eicosapentaenoic acid, salts of eicosapentaenoic acid, docosahexaenoic acid, salts of docosahexaenoic acid, triglycerides of eicosapentaenoic acid, tryglycerides of docosahexaenoic acid, ethyl esters of eicosapentaenoic acid, or ethyl esters of docosahexaenoic acid. In other embodiments, the polyunsaturated fatty acid includes at least one fish oil.

In some embodiments of the pre-concentrate, the oil phase includes a mixture of fish oils, or a mixture of at least one fish oil and Vitamin E. In certain additional embodiments, the ratio of fish oil to Vitamin E is between about 3:7 and about 9:1 or in other embodiments, the ratio of fish oil to vitamin E is between 3:7 and 1:1. In further embodiment of the present invention, the ratio of fish oil to Vitamin E is 7:3. In some instances, the combination of vitamin E and fish oil in the oil phase of the emulsion results in an additional reduction of the particle size of the emulsion (i.e. <50 nm) where the formulation can be sterile filtered.

In certain other embodiments of the pre-concentrate, the therapeutic agent is hydrophobic and is substantially miscible in the oil phase. In some embodiments, up to 50% of the oil phase is loaded with the therapeutic agent. In certain further embodiments, the therapeutic agent is one or more of an anti-inflammatory agent, analgesic, anti-allergenic, anti-fungal, anti-arrythmic agent, antibiotic, anticoagulant, antidepressant, antidiabetic agent, anti-epilepsi agent, anti-hypertensive agent, anti-gout agent, anti-malarial, anti-migraine agent, antimuscarinic agent, antineoplastic agent, anti-protozoal agent, anxiolytic, thyroid, anti-thyroid, antiviral, anoretic, bisphosphonate, cardiac inotropic agent, cardiovascular agent, corticosteroid, diuretic, dopaminergic agent, gastrointestinal agent, hemostatic, histamine receptor antagonist, hypnotic, immunosuppressant, kidney protective agent, lipid regulating agent, muscle relaxant, neuroleptic, neurotropic agent, opioid agonist, and antagonist, parsympathomimetic, protease inhibitor, prostaglandin, sedative, sex hormone, stimulant, sympathomimetic vasodilator, and xanthan, or mixtures thereof. In certain other embodiments, one or more of the therapeutic agents are selected from the group consisting of amphoteracin B, TAFA93, SAR943, ISA247, rapamycin, cyclosporine, cyclosporine A, other cyclosporine derivatives and rapamycin derivatives. In certain other embodiments of the pre-concentrate, the therapeutic agent is an anti-inflammatory agent such as a cyclosporine derivative. In certain embodiments, the cyclosporine derivative is ISA-247.

In certain embodiments, the pre-concentrate includes an emulsifier component which includes one or more surfactants. In certain examples, the surfactants are selected from the group consisting of Vitamin E TPGS, lecithin, SolutolHS-15, polysorbate 80, or cremophore EL. In certain other embodiments of the pre-concentrate, the surfactant is cremophore EL.

Another embodiment of the present invention provides a nanoemulsion pharmaceutical composition comprising a pre-concentrate as described herein and an aqueous medium, wherein the emulsifier component is less than about 10 wt % of the nanoemulsion pharmaceutical composition or less than 5 wt % of the nanoemulsion composition.

In certain embodiments of the nanoemulsion pharmaceutical composition, the oil phase is loaded up to 30% with the therapeutic agent or up to 50% with the therapeutic agent. In certain additional embodiments, the therapeutic agent is about 5 wt % of the nanoemulsion pharmaceutical composition. In certain other embodiments, the therapeutic agent is the cyclosporine derivative ISA-247. In yet other embodiments of the nanoemulsion pharmaceutical composition, the oil phase is between about 10 wt % and about 15 wt % of the nanoemulsion pharmaceutical composition.

In further embodiments, the aqueous medium of the nanoemulsion pharmaceutical composition includes water, glycofurol, ethyl acetate, propylene glycol, ethanol, lower alkanols, or mixtures thereof. In certain other embodiments, the aqueous medium is between about 75 wt % and about 85 wt % of the nanoemulsion pharmaceutical composition.

In certain other embodiments of the nanoemulsion pharmaceutical composition, the emulsifier component is between about 5 wt % and about 15 wt % of the nanoemulsion pharmaceutical composition. In another embodiment, the therapeutic agent is less than about 5 wt % of the nanoemulsion pharmaceutical composition.

In other embodiments of the nanoemulsion pharmaceutical composition, the nanoemulsion has a final therapeutic agent concentration of between about 20 mg/ml and about 60 mg/ml. In further embodiments, the final concentration of the therapeutic agent is between about 30 mg/ml and about 45 mg/ml.

In other embodiments of the nanoemulsion pharmaceutical composition, the nanoemulsion contains droplets having a diameter range between about 30 and about 200 nanometers. In other embodiments, the droplets have a diameter of less than about 120 nm. In further embodiments, the droplet size is les than 100 nm in diameter.

In certain embodiments, the nanoemulsion pharmaceutical composition is adapted for communication from an irrigation system to a body cavity, duct or surface.

In certain embodiments, the nanoemulsion pharmaceutical composition has an emulsion with a zeta-potential range from about 0 to about −40 meV. In certain other embodiments, the emulsion has a viscosity range from about 2.0 to about 3.0.

In yet further embodiments of the present invention, a nanoemulsion is provided as having (a) a pre-concentrate which includes (i) a mixture of fish oil and Vitamin E; (ii) a therapeutic agent; and (iii) cremophore EL; and (b) water; wherein the nanoemulsion has oil-in-water emulsion droplets and at least 50% of the emulsion droplets have a diameter of less than 100 nm; wherein the cremophore EL includes less than 5 wt % of the nanoemulsion; and the ratio of cremophore EL to the mixture of fish oil and Vitamin E is about 0.3:1 or less. In other embodiments, the therapeutic agent is ISA-247.

Yet another embodiment of the present invention provides a method of preparing a nanoemulsion formulation comprising (a) preparing a pre-concentrate as described herein to form a first mixture. The first mixture is added to an aqueous phase to form a second mixture. The method includes a third step of applying a microfluidizer to the second mixture to form a nanoemulsion. The method includes a fourth step in applying at least one PIT cycle to the first nanoemulsion to form a second nanoemulsion such that at least 70% of the emulsion droplets have a diameter of about 200 nm or less.

In certain other embodiments of the method of preparing the nanoemulsion formulation, 2 to 4 additional PIT cycles are applied to the second nanoemulsion. In further embodiments, the therapeutic agent is a cyclosporine derivative. In certain additional embodiments, the cyclosporine derivative is ISA-247.

In certain further embodiments of the method of preparing the nanoemulsion formulation, the therapeutic agent is at least about 20 wt % of the pre-concentrate or at least about 30 wt % of the pre-concentrate. In other embodiments of preparing the nanoemulsion formulation the second nanoemulsion pharmaceutical composition has a final therapeutic agent concentration of between about 20 mg/ml and about 60 mg/ml. In some embodiments, the final concentration of the therapeutic agent is between about 30 mg/ml and about 45 mg/ml.

In certain other embodiments, about 50% of the second nanoemulsion droplets have a diameter of less than about 100 nm.

Certain other embodiments of the method of preparing the nanoemulsion pharmaceutical composition, further includes the step of sterilizing the second nanoemulsion by passing the second nanoemulsion through a polycarbonate/PTFE 0.2 μm. In certain other embodiments, the oil phase includes a polyunsaturated fatty acid. In other embodiments of this invention the polyunsaturated fatty acid includes eicosapentaenoic acid, salts of eicosapentaenoic acid, docosahexaenoic acid, salts of docosahexaenoic acid, triglycerides of eicosapentaenoic acid, tryglycerides of docosahexaenoic acid, ethyl esters of eicosapentaenoic acid, and/or ethyl esters of docosahexaenoic acid. In additional embodiments, the polyunsaturated fatty acid is an omega-3 fatty acid. In certain other embodiments, the omega-3 fatty acid is a fish oil or a mixture of different fish oils. In other embodiments, the oil phase includes a mixture fish oil and vitamin E. In certain further embodiments, the ratio of fish oil to vitamin E is between about 3:7 and about 1:1.

In certain other embodiments of the method of preparing a nanoemulsion formulation, the ratio of the emulsifier component to the oil phase is about 0.3:1 or less.

Other embodiments of the method of preparing the nanoemulsion formulation is prepared such that the zeta-potential of the second nanoemulsion ranges from about 0 to about −40 meV. In other embodiments, the nanoemulsion has a viscosity range of about 2.0 to about 3.0.

Another embodiment of the present invention provides a method of administering to a subject a nanoemulsion pharmaceutical composition comprising the steps of a) integrating the nanoemulsion pharmaceutical composition, as described herein, with a medical or surgical device, and b) communicating the medical or surgical device to a target site of the subject, such that the nanoemulsion pharmaceutical composition is delivered to the target site. In certain embodiments, the medical or surgical device is an irrigation system. In certain embodiments, the irrigation system is a balloon catheter having a perforated or micro-porous portion. In certain further embodiments, the medical device is an IV bag. In certain embodiments, a therapeutically effective amount of the nanoemulsion pharmaceutical composition is applied to the irrigation system, a parenteral system, or a topical system and the device is communicated to a target site of the subject. In other embodiments, the target site is a body cavity, duct, surface or the like. In some embodiments, the irrigation system is a balloon catheter having a porosity greater than 0% and less than about 25%. In other embodiments, the irrigation system is adapted for communication to the target site. In certain other embodiments, the medical device is adapted for parenteral, mucosal, topical, or ocular administration to the target site.

In certain further embodiments, of the method of administrating the nanoemulsion pharmaceutical composition to a subject, the nanoemulsion is integrated with a micro-porous balloon portion of the balloon catheter via a lumen attached at a distal end to the balloon portion such that the nanoemulsion pharmaceutical composition traverses the pore of the balloon portion with a flow rate sufficient to effectively treat the target site.

### BRIEF DESCRIPTION OF THE DRAWINGS

The present invention will become better understood with reference to the following description and accompanying drawings, wherein:

FIG. 1 is a flow chart of the process of preparing the nanoemulsion pharmaceutical composition invention provided herein;

FIG. 2 is a flow chart depiction of the process of administering the nanoemulsion pharmaceutical composition to a target site of a subject;

FIG. 3 is a side cross-sectional view of a micro-porous medical device for use in delivery of the present invention; and

FIG. 4 is a schematic representation of the microstructure of a section of the wall of the micro-porous medical device.

### DETAILED DESCRIPTION

Prior to discussing the present invention, several definitions of terms utilized herein to describe the invention are provided below.

The phrases “emulsifying component,” and “surfactant” are used interchangeably herein to refer to the component used to stabilize the emulsions provided herein.

As used herein, the term “therapeutically effective” refers to a dose that produces the desired effect for which it is administered. The exact dose will depend on the purpose of the treatment, and will be ascertainable by one skilled in the art using known techniques. Efficacy can be measured in conventional ways, depending on the condition to be treated. Therapeutically effective amount also refers to a target serum concentration, such as a trough serum concentration, that has been shown to be effective in suppressing disease symptoms when maintained for a period of time.

As used herein, the phrase “poorly water-soluble” shall refer to a material that is not soluble in water, at 25° C., at a concentration of about 0.2% by weight, and preferably not soluble at about 0.1% by weight.

For purposes of this specification and the accompanying claims, the term “irrigation system” refers to any device or system used to deliver at least water, but possibly also nutrients, and other materials to a target site as defined herein. An irrigation system includes a supply of water, a delivery mechanism and any necessary conduits or tubes necessary to conduct the water to target site(s). An irrigation system may further include additional components, including, but not limited to, a filtration system, a sterilization system, and a de-ionizing component.

The term “porosity” as used herein refers a degree to which a balloon that contains micro-pores is porous to liquids having pre-defined particle size characteristics. One measure of porosity is the water entry pressure (“WEP”) test. This test records the pressure at which water supplied to the interior chamber of a balloon begins to weep out the pores in the balloon walls. Another method used to measure porosity is the bubble point. In addition, porosity can be indicated as a measurement of the internodal distance between nodes forming the material of the balloon walls, as is understood by those of ordinary skill in the art.

As used herein, the term “shear homogenization” is a process in which two or more phases are mixed together under intense external forces, whereas under normal conditions they are not miscible. The high speed rotation of the homogenizer subjects the mixture to intense mechanical and hydraulic shear. Materials are rapidly mixed dispersed and emulsified, resulting in efficient droplet-size reduction and uniform, constant final products mixtures. The term “shear” is to be broadly construed to include shearing and agitation provided by a wide variety of mixing and/or emulsifying devices. The size of various sets of droplets so prepared is inversely proportional to the rate of shear employed (i.e., the higher the shear rate, the smaller the droplet size). It is also understood that droplet sizes are also affected by factors such as the type of blade employed in the device providing the high shear rate, and the like.

The term “droplets” is used herein to denote the dispersed phase of the emulsions provided herein containing the therapeutic agent.

As used herein, the term “micro-porous” refers to porous films, membranes or film layers having an average pore size of 0.05 to 3.0 microns as measured by bubble point pore size ASTM-F-316-80.

Turning to the present invention; compositions having emulsion droplets with an average diameter of less than or equal to about 200 nm (“nanoemulsion”) have improved stability and/or activity. Such compositions further having therapeutic activity are particularly well suited for therapeutic or cosmetic use. The present invention is directed toward nanoemulsion formulations of poorly water-soluble therapeutic agents where the nanoemulsions have sub-micron sized droplets and improved toxicity. The nanoemulsions are prepared by low energy methods that require less surfactant relative to conventional nanoemulsions. The present invention is also directed toward methods of preparing the nanoemulsions and methods of locally administering the emulsified poorly water-soluble therapeutic agents to a subject, using a medical or surgical device that has been integrated with the nanoemulsion. It has been surprisingly found that the toxicity, solubility and bioavailability of poorly water-soluble therapeutic agents can be improved by nanoemulsion formulations prepared using less surfactants and less energy than conventional nanoemulsions. Nanoemulsions of the present invention include a pre-concentrate and an aqueous phase. The methods of preparing the nanoemulsions of the present invention include a modified phase inversion temperature method (PIT). The present invention also provides methods of administering the nanoemulsions provided herein by incorporating the nanoemulsion with a medical or surgical device that can be used to locally deliver the nanoemulsion to a target site of the subject.

Preparation of the pre-concentrate is an important step in the preparation of the nanoemulsion. In accordance with the present invention, a pre-concentrate, including a hydrophobic therapeutic agent, is formed and combined with an aqueous medium to prepare the desired nanoemulsion.

### Pre-Concentrate

The present invention provides a pre-concentrate which includes an oil phase, a therapeutic agent and an emulsifier component, where the ratio of the emulsifier component to the oil phase is about 1:1 or less. It has been surprisingly found that nanoemulsions can be prepared using pre-concentrates that use less surfactant to achieve the nanoemulsion than conventional nanoemulsions. A pre-concentrate is prepared and later integrated with an aqueous phase to form an emulsion.

The pre-concentrate includes an oil phase which has at least one fatty acid oil. Fatty acid oils of the present invention include at least one polyunsaturated fatty acid. The term “polyunsaturated fatty acid” as used herein shall include those fatty acids having at least 50 weight percent or more of polyunsaturated fatty acids. Polyunsaturated fat can be found in grain products, fish and sea food (herring, salmon, mackerel, halibut), soybeans, and fish oil. Polyunsaturated fatty acids include omega-3 fatty acids and omega-6 fatty acids. Polyunsaturated fatty acids include linolic acid, linolenic acid and the like. Preferable polyunsaturated fatty acids include eicosapentaenoic acid, salts of eicosapentaenoic acid, docosahexaenoic acid, salts of docosahexaenoic acid, triglycerides of eicosapentaenoic acid, tryglycerides of docosahexaenoic acid, ethyl esters of eicosapentaenoic acid, or ethyl esters of docosahexaenoic acid.

Fatty acid oil refers to an oil or fat comprised predominantly of fatty acids. Fatty acids are monobasic organic acids which are derived from natural fats and oils but can be made synthetically. Generally they have the formula CₙH₂ₙ₊₁COOH. Those acids whose molecules have an even number of carbon atoms (usually 8 to 22 but can be longer or shorter in chain length) arranged in a straight chain are by far the most common and may be either saturated or unsaturated. The most abundant acids have 16 or 18 carbon atoms and these are commercially the most important.

Polyunsaturated fatty acids include, among several others, omega-3 fatty acid oils and medium chain triglycerides (MCT). A medium chain triglyceride contains about 6 to 14 carbon atoms, preferably about 8 to 12 carbon atoms are suitable for use in the oil phase. Preferable medium chain glyceride includes, for example, caprylic/capric triglyceride such as “Migriol 810”, “Migriol 812” (both trade names, manufactured by Huls Co., Ltd., available from Mitsuba Trading Co., Ltd.), a glyceryl tricaprylate (tricaprylin) such as “Panasate 800” (trade name, manufactured by NOF Corporation, Japan) and the like.

Omega-3 fatty acid oils are found, for example, in fish oils. The pre-concentrate of the present invention, in certain embodiments, contains at least one fish oil that is rich in omega-3 fatty acid oils. Omega-3 fatty acid oils includes a natural or synthetic omega-3 fatty acid, and pharmaceutically acceptable esters, derivatives, precursors or salts thereof and mixtures thereof. Examples of omega-3 fatty acid oils include but are not limited to omega-3 polyunsaturated, long-chain fatty acids such as a eicosapenta-5,8,11,14,17-enoic acid (“EPA”), docosahexa-4,7,10,13,16,19-enoic acid (“DHA”), and .alpha.-linolenic acid; esters of an omega-3 fatty acid with glycerol such as mono-, di- and triglycerides; esters of the omega-3 fatty acid and a primary alcohol such as fatty acid methyl esters and fatty acid ethyl esters; precursors of an omega-3 fatty acid oil, such as EPA and DHA precursor .alpha.-linolenic acid; and derivatives such as polyglycolized derivatives or polyoxyethylene derivatives. Preferred omega-3 fatty acid oils are EPA or DHA, triglycerides thereof, ethyl esters thereof and mixtures thereof. The omega-3 fatty acids or their esters, derivatives, precursors, salts and mixtures thereof can be used either in their pure form or as a component of an oil such as fish oil (otherwise known as marine oil), preferably highly purified fish oil concentrates, or perilla oil or marine microalgae oil. Suitable fish oils are, for example, those types which are recovered in substantial quantities from cold-water fish, such as pilchard oil, menhaden oil, Peruvian fish oil, sardine oil, salmon oil, herring oil, and mackerel oil. Preferably, the fish oil has a high omega-3 fatty acid oil content, such as 50% or higher, more preferably, 70% or higher, most preferably 80% or higher.

In certain aspects of the present invention, the oil phase is a blend of native omega-3 fatty acid rich fish oil and alpha-tocopherol (Vitamin E, VE). The fish oil and the VE are admixed together in ratios ranging from between 3:7 to 7:3. In certain aspects the admixture or blend produces a ratio of fish oil to Vitamin E of 7:3. The ratio of the native fish oil to Vitamin E have been surprisingly found to be important in the size of the droplets achieved by the phase inversion temperature (PIT) method when the pre-concentrate is integrated with an aqueous phase. Specifically, emulsions prepared with a combination of fish oil and vitamin E in the oil phase of the emulsion can result in the formation of particles less than 100 nm, and preferably less than 50 nm.

The pre-concentrate includes an emulsifier component. The emulsifier component has one or more surfactants. Surfactants include any molecule having both a polar head group, which energetically prefers solvation by water, and a hydrophobic tail that is not well solvated by water. The ratio of the oil phase to the emulsifier component is important for the toxicity of the nanoemulsion prepared from the pre-concentrate. A variety of surfactants are contemplated including, but not limited to, nonionic and ionic surfactants. While the present invention is not limited to any particular mechanism, it is contemplated that surfactants, when present in the pre-concentrate or in the emulsion, helps to stabilize the emulsions. Both non-ionic and ionic surfactants are contemplated.

Additionally, surfactants from the BRIJ family of surfactants find use in the compositions of the present invention. The surfactant can be provided in either the aqueous or the oil phase. Surfactants suitable for use with the pre-concentrate and emulsion include a variety of anionic and nonionic surfactants, as well as other emulsifying compounds that are capable of promoting the formation of oil-in-water emulsions; so long as they are on the GRAS (Generally Recognized as Safe) list and are approved for human consumption such as lecithin, solutol HS-15 (polyoxyethylene esters of 12-hydroxystearic acid), polysorbate 80 or Cremophore EL (polyethoxylated castor oil). In general, emulsifying compounds are relatively hydrophilic, and blends of emulsifying compounds can be used to achieve the necessary qualities. In some formulations, nonionic surfactants have advantages over ionic emulsifiers in that they are substantially more compatible with a broad pH range and often form more stable emulsions than do ionic (e.g., soap-type) emulsifiers. Thus, in certain preferred embodiments, the compositions of the present invention comprise one or a blend of two or more non-ionic surfactants. The non-ionic surfactants which are particularly preferred in the invention are selected especially from polyethoxylated hydrogenated castor oil containing 35 mol of ethylene oxide (hereafter referred to as “with 35 EO”), polyethoxylated hydrogenated castor oil containing 7 mol of ethylene oxide (or with 7 EO), polyethoxylated olive oil with 7 EO, sorbitan monooleates with 4 EO, 5 EO or 20 EO, (C.sub.12-C.sub.14-alkyl)glycosides or (C.sub.8-C.sub.14-alkyl)glycosides, glycerol monostearate with 30 EO, decaglyceryl monooleate, polyalkoxylated oleyl alcohol with 2 or 10 EO, polyethoxylated lauryl alcohol with 7 EO, methylglucoside dioleate, and mixtures thereof. Polyethoxylated castor oil-based emusifiers, such as cremophore EL or the less sensitizing solutol HS-15, are most preferred. In certain aspects of the present invention, the surfactant makes up less than 10% of the total nanoemulsion composition. In certain aspects, long chain ethoylated surfactants such as Cremophore EL 35, polyethoylated castor oil, a BASF product, TPGS1000, polyethyleneglycol 1000 ester of alpha-tocopheryl succinate, TWEEN 80, or polyoxyethylene 20 sorbitan monooleate may also be employed as the surfactant of choice.

In certain aspects Vitamin E TPGS may also be used in the surfactant blend. TPGS or PEGylated vitamin E is a vitamin E derivative in which polyethylene glycol subunits are attached by a succinic acid diester at the ring hydroxyl of the vitamin E molecule. TPGS stands for D-.alpha.-tocopherol polyethyleneglycol 1000 succinate (MW=530). TPGS is a non-ionic surfactant having an HLB value between 16 and 18.

HLB as used herein refers to the Hydrophile-Lipophile Balance Index Number and is an index for correlating the chemical structure of surfactant molecules with their surface activity. The HLB Index Number may be calculated by a variety of empirical formulas as described by Meyers, (Meyers, Surfactant Science and Technology, VCH Publishers Inc., New York, pp. 231-245 [1992]), incorporated herein by reference. As used herein, the HLB Index Number of a surfactant is the HLB Index Number assigned to that surfactant in McCutcheon's Volume 1: Emulsifiers and Detergents North American Edition, 1996 (incorporated herein by reference). The HLB Index Number ranges from 0 to about 20 or more for commercial surfactants. Hydrophilic surfactants with high solubility in water and solubilizing properties are at the high end of the scale, while surfactants with low solubility in water that are good solubilizers of water in oils are at the low end of the scale.

Important to the toxicity and droplet size, and therefore to the solubility and bioavailability, of the emulsified therapeutic agent is the ratio of the oil phase to the emulsifier components in the pre-concentrate and the nanoemulsion. While not wishing to be bound by any specific theory, it has been found and is believed that the droplet size of the nanoemulsion is dependent on the critical surfactant or surfactant blend concentration. In other words, the droplet size depends more on the surfactant-to-oil weight ratio rather than by the aqueous phase content in the nanoemulsion. In some embodiments of the invention, the ratio of the oil phase to the emulsifier component in the present invention is 0.4:1. In other embodiments of the invention, the ratio is 0.3:1.

The pre-concentrate also includes a therapeutic agent. A therapeutic agent includes any composition that decreases the infectivity, morbidity, or onset of mortality in a host contacted by a pathogen or that prevents infectivity, morbidity, or onset of mortality in a host contacted by a pathogen. Such agents include a poorly water-soluble therapeutic agent or a mixture of poorly water-soluble therapeutic agents that can be beneficially co-administered with an omega-3 fatty acid oil to a mammal, especially a human. By “poorly water-soluble therapeutic agent” what is meant is that the therapeutic agent is insoluble in water or has an aqueous solubility of less than about 5 parts per 1000 parts of water by weight at 20° C. Poor water-soluble therapeutic agent of the present invention includes cyclosporine and cyclosporine derivatives such as ISA-247.

Alternative therapeutic agents may include a blend of two or more therapeutic agents. Additionally, the therapeutic agent may be hydrophilic and may be incorporated in the aqueous phase of the nanoemulsion rather than in the oil phase of the pre-concentrate. Other therapeutic agents included in the pre-concentrate, besides ISA-247, may also include one or more of an anti-inflammatory agent (i.e., any cyclosporine derivative), analgesic, anti-allergenic, anti-fungal, anti-arrythmic agent, antibiotic, anticoagulant, antidepressant, antidiabetic agent, anti-epilepsi agent, anti-hypertensive agent, anti-gout agent, anti-malarial, anti-migraine agent, antimuscarinic agent, antineoplastic agent, anti-protozoal agent, anxiolytic, thyroid, anti-thyroid, antiviral, anoretic, bisphosphonate, cardiac inotropic agent, cardiovascular agent, corticosteroid, diuretic, dopaminergic agent, gastrointestinal agent, hemostatic, histamine receptor antagonist, hypnotic, immunosuppressant, kidney protective agent, lipid regulating agent, muscle relaxant, neuroleptic, neurotropic agent, opioid agonist, and antagonist, parsympathomimetic, protease inhibitor, prostaglandin, sedative, sex hormone, stimulant, sympathomimetic vasodilator, and xanthan, or mixtures thereof. In certain other embodiments, one or more of the therapeutic agents are selected from the group consisting of amphoteracin B, TAFA93, SAR943, rapamycin, cyclosporine, cyclosporine A, other cyclosporine derivatives and rapamycin derivatives.

In other aspects of the present invention, the therapeutic agent is hydrophobic and is therefore substantially miscible in the oil phase of the pre-concentrate and the nanoemulsion. The therapeutic agent is loaded into the oil phase. The oil phase has in certain embodiments a mixture of fish oil and Vitamin E in a 7:3 ratio, though other oil phase compositions are contemplated. The therapeutic agent, such as ISA-247, is added to the oil phase, such as the fish oil Vitamin E blend, and is incorporated into the oil phase by being vigorously vortexed at about 130 degrees C. for about 2 minutes. This particular method of integrating the therapeutic agent into the oil phase allows for the therapeutic agent to be substantially loaded into the oil phase. In certain aspects of the invention, the therapeutic agent is loaded into the oil phase such that up to 30% of the oil phase contains a therapeutic agent or blend of therapeutic agents. In certain other aspects the vortex is applied to the oil phase-therapeutic agent blend such that the oil phase is loaded up to 50% with the therapeutic agent to create a drug-containing oil phase. The drug containing oil phase can then be integrated with the emulsifier component to form the pre-concentrate.

The pre-concentrate is formed by loading the oil phase with the therapeutic agent to form a drug-containing oil phase. The emulsifier component is added to the drug-containing oil phase. Other sequences are also contemplated. For example, the therapeutic agent can be loaded into one or more of the surfactants of the emulsifier component to create a drug-containing emulsifier component. The drug-containing emulsifier component is then integrated with the oil phase.

Now having discussed the pre-concentrate, attention is now directed to the creation of a nanoemulsion with the pre-concentrate heretofore described.

### The Nanoemulsion

An emulsion is a composition containing an aqueous phase and an oil phase stabilized by a surfactant. The term “emulsion,” refers to, without limitation, any oil-in-water dispersions or droplets, including lipid structures that can form as a result of hydrophobic forces that drive apolar residues (e.g., long hydrocarbon chains) away from water and polar head groups toward water, when a water immiscible phase is mixed with an aqueous phase. These other lipid structures include, but are not limited to, unilamellar, paucilamellar, and multilamellar lipid vesicles, micelles, and lamellar phases. Nanoemulsions of the present invention are emulsions containing droplets that have an average diameter of less than about 200 nm.

The present invention provides a nanoemulsion pharmaceutical composition (“nanoemulsion”). The nanoemulsion includes a pre-concentrate, as described herein, and an aqueous medium. The emulsifier component comprises less than about 10 wt % of the nanoemulsion. The pre-concentrate as described herein is integrated with the aqueous phase by mixing mechanisms. In certain embodiments, the mixture of the pre-concentrate and the aqueous phase spontaneously forms an emulsion. Reducing the droplet sizes down to an average diameter of less than 200 nm requires further processing. Further processing includes implementation of a phase inversion temperature (PIT) method. The PIT method may be used in conjunction with some high-shear stiffing such that less energy is used than would be utilized if high-shear stiffing was used alone. In certain embodiments the PIT method is used instead of the high-shear mechanism. The high shear-stirring may be by way of MICROFLUIDIZER® Processor (homogenizer). In general, the smaller the droplet diameter, the more high energy input is required by the high-shear stiffing if used alone. The high-energy input leads to deforming forces that are able to break the droplets into smaller ones, provided that the Laplace pressure is overcome. An increase in the surfactant content at the interface of the phases may also reduce the Laplace pressure making it easier to overcome. However, high surfactant content leads to higher toxicity.

By the present invention, less surfactant and less high energy high-shear stirring is used to reduce the droplet size. In certain aspects of the invention, the emulsifier component is between about 5 wt % and about 15 wt % of the nanoemulsion pharmaceutical composition. The emulsifier preferably comprises less than 5 wt % of the nanoemulsion pharmaceutical composition. While more surfactant would further stabilize the nanoemulsion, an increase in surfactant content leads to increased toxicity. While not wishing to be bound by any specific theory, the present invention reduces toxicity by reducing the surfactant content in the pre-concentrate and in the nanoemulsion.

In certain aspects of the present invention, the aqueous medium is between about 75 wt % and about 85 wt % of the nanoemulsion pharmaceutical composition. The pre-concentrate or the nanoemulsion can be further diluted without affecting the droplet size. The aqueous medium of the nanoemulsion is water or water admixed with lower alcohols. The aqueous medium may alternatively be a water-soluble alcohol.

Alternatively, the aqueous medium of the nanoemulsions can be either purified or ultrapure water, saline, buffered saline, buffered aqueous medium, glycerine, low molecular weight polyethylene glycol either alone or in combinations thereof. The concentration of aqueous medium in nanoemulsions can vary from 30% to 90%. Preferably, the aqueous medium of the present invention is between about 75 wt % and about 85 wt %. The aqueous medium may alternatively include glycofurol, ethyl acetate, propylene glycol, ethanol, lower alkanols, or mixtures thereof.

Examples of the water-soluble alcohols include methanol, ethanol, propanol, isopropanol, ethylene glycol, propylene glycol, 1,3-butylene glycol, glycerol, sorbitol, mannitol, diethylene glycol, dipropylene glycol, polyethylene glycol (molecular weight, 400 to 20,000), sorbitan, sorbitol, maltose, maltotriose and sodium hyaluronate. Examples of preferred aqueous mediums include Glycofurol, ethyl acetate, propylene glycol, ethanol, lower alkanols, or mixtures thereof. Other suitable alcohols include, for example, C₁-C₁₂ alcohols, diols, and triols, for example glycerol, methanol, ethanol, propanol, octanol, and combinations comprising one or more of the foregoing alcohols. In one embodiment, the alcohol is ethanol or glycerol, or combinations thereof.

The aqueous phase ranges from a pH of about 4 to about 10. In one embodiment the pH of the aqueous phase ranges from about 6 to about 8. The pH of the aqueous phase can be adjusted by addition of an acid or a base such as, for example, hydrochloric acid or sodium hydroxide. In one embodiment, the aqueous phase is deionized water or distilled water.

The pre-concentrate, integrated into the aqueous phase, in certain aspects has an oil phase that is loaded up to 30% with the therapeutic agent. Alternative embodiments of the invention provide an oil phase that is vortexed with the therapeutic agent such that up to 50% of the oil phase is integrated with the therapeutic agent to form a drug-containing oil phase. Relative to the nanoemulsion pharmaceutical composition the therapeutic agent, which in preferred embodiments is hydrophobic and is substantially miscible in the oil phase, is about 5 wt % of the nanoemulsion pharmaceutical composition. The therapeutic agent is ISA-247 though other therapeutic agents are contemplated as herein previously, or subsequently discussed.

In certain aspects of the nanoemulsion pharmaceutical composition, the oil phase is 10-15 wt % of the nanoemulsion while the emulsifier component is 1-10 wt % of the nanoemulsion. The aqueous phase ranges from 75-85 wt % of the nanoemulsion. The nanoemulsion is prepared by a process which includes the PIT method and in conjunction with the vortex method. The vortex method is used integrate the therapeutic agent with the oil phase such that the oil phase is loaded up to 30 wt % or more preferably up to 50 wt %. It has surprisingly been found that when the pre-concentrate having the drug-containing oil phase is integrated into the final nanoemulsion (pre-concentrate & aqueous phase), the nanoemulsion reaches a final concentration of up to 45 mg/ml. Other concentrations are also contemplated and are provided herein by this invention such as a 30 mg/ml concentration of the therapeutic agent. The invention contemplates a final concentration range of the therapeutic agent that is between about 30 mg/ml and about 45 mg/ml. In other aspects the vortex method is used to load the oil phase with the therapeutic agent such that the nanoemulsion has a final therapeutic agent concentration of between 20 mg/ml and about 60 mg/ml. These concentrations allow for optimization of each of the formulation steps separately to achieve the best control of the nanoemulsion formulation. The calculated recovery of the therapeutic agent in the nanoemulsion pharmaceutical composition is over 90%.

Examples of suitable therapeutic agents include nephrotoxic drugs such as cyclosporines, cyclosporine analogs such as ISA-247; TAFA93, SAR943, rapamycin, cyclosporine A, rapamycin derivatives and amphotericin B; cardiotoxic drugs such as amphotericin B and FK506; drugs with immunosuppressive effects or anti-inflammatory drugs such as drugs for treating rheumatology, arthritis, psoriasis, inflammatory bowel disease, Crohn's disease or demyelinating diseases including multiple sclerosis; anti-tumor drugs such as melphalan, chlormethine, extramustinephosphate, uramustine, ifosfamide, mannomustine, trifosfamide, streptozotocin, mitobronitol, methotrexate, fluorouracil, cytarabine, tegafur, idoxide, taxol, paclitaxel, daunomycin, daunorubicin, bleomycin, amphotericin; hyperlipidemia or hypercholestolemia drugs such as fenofibrate; dioplar disease drugs; drugs which increase lipids and/or triglyceride levels; and drugs for treating Alzheimer's disease. The therapeutic agent can be selected from a variety of known classes of drugs including, but not limited to, analgesics, anti-allergic agents, anti-fungals, anti-inflammatory agents, antidepressants, agents, The therapeutic agent may include a mixture of poorly water-soluble drugs that can be beneficially co-administered with an omega-3 fatty acid oil. The therapeutic agent can also be selected from one or more of an anti-inflammatory agent, analgesic, anti-allergenic, anti-fungal, anti-arrythmic agent, antibiotic, anticoagulant, antidepressant, antidiabetic agent, anti-epilepsi agent, anti-hypertensive agent, anti-gout agent, anti-malarial, anti-migraine agent, antimuscarinic agent, antineoplastic agent, anti-protozoal agent, anxiolytic, thyroid, anti-thyroid, antiviral, anoretic, bisphosphonate, cardiac inotropic agent, cardiovascular agent, corticosteroid, diuretic, dopaminergic agent, gastrointestinal agent, hemostatic, histamine receptor antagonist, hypnotic, immunosuppressant, kidney protective agent, lipid regulating agent, muscle relaxant, neuroleptic, neurotropic agent, opioid agonist, and antagonist, parsympathomimetic, protease inhibitor, prostaglandin, sedative, sex hormone, stimulant, sympathomimetic vasodilator, and xanthan, or mixtures thereof.

The emulsifier component is used to lower the interfacial tension to solubilize of the immiscible mixture of the oil phase and the aqueous phase to solubilize these components. Because different drugs have different physicochemical properties different formulations are required for different drugs. For example, for certain drugs in nanoemulsions a surfactant or surfactant blend which has a HLB value of between 12 and 15 is ideal. It is important to have a formulation that is suitable for drugs having various physicochemical properties. In certain embodiments of the invention the surfactant or blend of surfactants employed in the pre-concentrate includes certain long-chain ethoylated surfactants, such as Cremophore EL 35, Polyetholylated castor oil, TPGS1000, polyethylene glycol 1000 ester of alpha-tocopheryl sucinate; Tween 80, polyoxyethylene 20 sorbitan monooleate.

Surface rheological properties which are affected by surfactant directly influence the diffusion of the emulsified therapeutic agent. Due to the physico-chemical properties and structures of surfactants, it can increase permeation and facilitate delivery of the therapeutic agent. Different therapeutic agents have different physico-chemical properties and therefore require different formulations for their intended use.

Surprisingly, a critical surfactant concentration has been found whereby a single formulation may be applied to different drugs, having different physicochemical properties. The droplet size of the formulation does not significantly depend on the amount of water added to the system. Rather, the influence of the critical concentration of the surfactant blend is more influential to the droplet size.

The nanoemulsion pharmaceutical composition is prepared by reducing the droplet size using the PIT method. The droplet size of the nanoemulsion is reduced such that the droplets have an average diameter of between about 100 nm and about 120 nm. Droplet sizes of less than about 120 nm are contemplated, along with all specific values and sub ranges therein. Adjusting the temperatures, pressures and varying the surfactant blend and the fish oil blend and employing additional cycles of heating and cooling, the droplet size is reduced to between 25 to 30 nm. Certain aspects of the present invention provide a nanoemulsion having droplets with average diameters of between 25 to 30 nm.

Certain aspects of the present invention contemplate and provide a nanoemulsion pharmaceutical composition that is adapted for communication from an irrigation system or any medical device to a body cavity. Local direct treatment is contemplated by placing the medical device in contact with the tissue to be treated by the emulsified therapeutic agent that has been integrated into the nanoemulsion pharmaceutical composition. The medical device may be an irrigation system such as a catheter or more specifically a balloon catheter, wherein the droplet sizes are small enough to pass through a micro-porous portion of the catheter.

One useful parameter for characterizing a nanoemulsion is “zeta potential”. Zeta potential is the electrical potential of a shear plane (an imaginary surface separating a thin layer of liquid that shows elastic behavior) bound to a solid surface that shows normal viscous behavior. The stability of hydrophobic colloids depends, in part, on the zeta potential. Zeta potential of a nanoemulsion can be about −50 meV to about +50. In one embodiment, the zeta potential of the emulsions range from about 0 to about −40 meV and a viscosity range from about 2.0 to about 3.0.

In other aspects of the invention a nanoemulsion is provided wherein a pre-concentrate has a mixture of fish oil and Vitamin E and a therapeutic agent and cremophore EL. The nanoemulsion can further have water as an aqueous phase and the nanoemulsion has oil-in-water emulsion droplets with at least 50% of the emulsion droplets have a diameter of less than 100 nm. The Cremophore EL is less than 5 wt % of the nanoemulsion and the ratio of Cremophore EL to the mixture of fish oil and Vitamin E is about 0.3:1. This nanoemulsion pharmaceutical composition is prepared as discussed above with a hydrophobic therapeutic agent being incorporated into the oil phase by vigorous vortexing. The example therapeutic agent used in this embodiment is ISA-247.

Nanoemulsion compositions can further contain various additives. Exemplary additives include, for example, activity modulators, gelling agents, thickeners, auxiliary surfactants, other agents that augment cleaning and aesthetics, and combinations comprising at least one of the foregoing, so long as they do not significantly adversely affect the activity and/or stability of the emulsions. Additives can be incorporated into the nanoemulsion or formulated separately from the nanoemulsion, i.e., as a part of a composition containing a nanoemulsion.

Activity modulators are additives that affect the activity of a nanoemulsion against the target microorganism. Exemplary activity modulators are interaction enhancers such as germination enhancers, therapeutic agents, buffers, and the like, which are described below.

One class of activity modulators thus includes interaction enhancers, compounds, or compositions that increase the interaction of the nanoemulsion with the cell wall of a bacterium (e.g., a Gram positive or a Gram negative bacteria) or a fungus, or with a virus envelope. Again, without being bound by theory, it is proposed that the activity of the emulsions is due, in part, to the interaction of a nanoemulsion with a microorganism membrane or envelope. Suitable interaction enhancers include compounds that increase the interaction of the nanoemulsion with the cell wall of Gram negative bacteria such as Vibrio, Salmonella, Shigella, Pseudomonas, Escherichia, Klebsiella, Proteus, Enterobacter, Serratia, Moraxella, Legionella, Bordetella, Helicobacter, Haemophilus, Neisseria, Brucella, Yersinia, Pasteurella, Bacteiods, and the like.

A nanoemulsion can be prepared in a diluted or an undiluted form. In one embodiment of the present invention, a nanoemulsion shows suitable stability in both diluted and undiluted forms. By suitable stability, it is meant that the emulsions do not show any signs of separation (oil phase from aqueous phase) for at least 6 months. In one embodiment of the present invention, a nanoemulsion does not show any sign of separation up to about 2 years. In one embodiment of the present invention, a nanoemulsion does not show any sign of separation for up to about 3 years In another embodiment of the present invention, a nanoemulsion does not show any sign of separation up to about 2 years. In a further embodiment of the present invention, a nanoemulsion does not show any sign of separation for up to about 3 years. Settling of the diluted emulsions is an acceptable characteristic and does not indicate separation of an oil phase from an aqueous phase. Settling is due to separation of emulsions from its diluent, not an oil phase separating from an aqueous phase. Such settling is readily reversed by simple shaking of the nanoemulsion, while separation of the concentrated emulsions are not reversed by simple mixing, requiring instead re-emulsification.

The emulsions can also contain a first nanoemulsion emulsified within a second nanoemulsion, wherein the first and second emulsions can each contain an aqueous phase, an oil phase, and a surfactant. Either one or both nanoemulsions of this composition can contain an anti-inflammatory agent. The oil phase of each of the first and second nanoemulsion can contain an oil and an organic solvent. The first and second nanoemulsion can be the same or different. A nanoemulsion can also contain a first nanoemulsion re-emulsified to form a second nanoemulsion.

### Method of Preparation

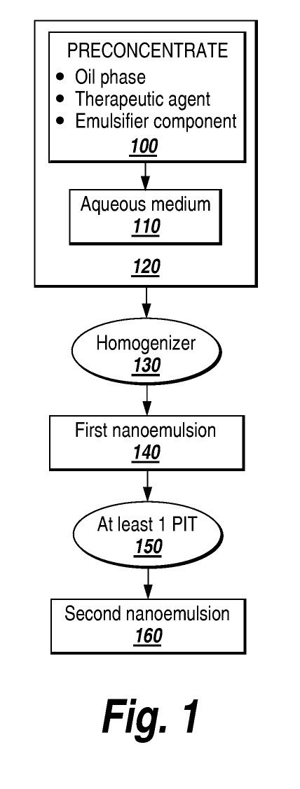

Other aspects of the invention provided herein include a method of preparing the nanoemulsions provided herein. In certain non-exhaustive aspects the method, as depicted in flow chart of FIG. 1, includes the following steps: preparing the pre-concentrate (100) provided herein to form a first mixture (100); adding the first mixture to an aqueous (110) phase to form a second mixture (120); and, applying a high shear-stirring mechanism (130), such as a microfluidizer, to the second mixture to form a first nanoemulsion (140); applying at least one PIT cycle (150) to the first nanoemulsion to form a second nanoemulsion, (160) such that at least 70% of the emulsion droplets have a diameter of about 200 nm or less. Incorporating the PIT method into the process allows further reduction of the size of the nanoemulsion droplets without having to employ further high-energy high-shear stiffing mechanisms. Additional PIT cycles may also be employed to further reduce the droplet size to less than 120 nm.

The PIT method achieves nanoemulsions with reduced droplet sizes by employing the physicochemical properties of the system. This is a low-energy emulsification method. This method makes use of changing the spontaneous curvature of the surfactant. For non-ionic surfactants, this can be achieved by changing the temperature of the system, forcing a transition from an oil-in-water (O/W) emulsion at low temperatures to a water-in-oil (W/O) at higher temperatures (transitional phase inversion). During cooling, the system crosses a point of zero spontaneous curvature and minimal surface tension, promoting a formation of finely dispersed oil.

Additionally, instead of temperature, other parameters such as salt concentration or pH value may be adjusted as well, generalized by considering the surfactant affinity difference (SAD) instead of the temperature alone. Moreover, a transition in the spontaneous radius of curvature can be obtained by changing the water volume fraction (emulsion inversion point (EIP) method). By successively adding water into oil, initially water droplets are formed in a continuous oil phase. Increasing the water volume fraction changes the spontaneous curvature of the surfactant from initially stabilizing a W/O emulsion to an O/W emulsion at the inversion locus. Also during this transition, referred to as catastrophic phase inversion (CPI), minimal interfacial tensions are achieved and reported to facilitate the formation of fine droplets.

In certain aspects, the PIT method comprises steps such as heating water to a temperature greater than a phase inversion temperature of an emulsifier used in the nanoemulsion; heating an oil phase of the nanoemulsion in a separate vessel to the same temperature as the water, wherein the oil phase comprises at least the emulsifier and a lipophilic material; adding the heated oil phase to the heated water to obtain a mixture; and cooling the mixture to a temperature below the phase inversion temperature of the emulsifier to form the nanoemulsion.

A small droplet size nanoemulsion can be formed in the first instance or can be formed from a nanoemulsion having larger droplets. For example, a small droplet size nanoemulsion can be produced by reducing the droplet size of a classical or conventional nanoemulsion (i.e., above 250 nm droplet size), to produce a nanoemulsion wherein the average nanoemulsion droplet size is less than about 200 nm. In other words, a nanoemulsion having an average droplet diameter of greater than about 250 nm is treated in a manner effective to produce droplets having an average diameter of less than or equal to less than 200 nm. In one embodiment, small droplet size nanoemulsion droplets have an average diameter of less than or equal to about 200 nm, less than or equal to about 120 nm, less than or equal to about 100 nm, less than or equal to about 50 nm, and less than or equal to about 30 nm.

The droplet size in the nanoemulsion can be determined using any means known in the art, such as, for example, using laser light scattering.

Once the heated vortex method has been applied to the oil phase and the therapeutic agent has been incorporated into the oil phase to form the drug-containing oil phase, the drug-containing oil phase is then placed in contact with an emulsifier component to form the pre-concentrate. The pre-concentrate, which includes the oil phase, is poured into the aqueous medium. The emulsifier component includes at least one surfactant or a blend of surfactants. Preferably, the surfactants are non-ionic. It has been surprisingly found that the ratio of emulsifier phase to oil phase is the critically element which determines the size of the nanoemulsion droplets prepared by the Phase Inversion Temperature (PIT) method. The amount of the aqueous phase is not determinative of the droplet size. While not wishing to be bound by any specific theory, it is believed that the droplet size is dependent on the distance given by the lamellar structure at the inversion point. In certain embodiments it has been found that using the PIT method allowed the nanoemulsion to be formed with less surfactant. For example in certain embodiments of the invention, nanoemulsion droplets having droplet sizes less than 200 nm were prepared where the ratios of the emulsifier component to fatty acid oil is about 1:1, and 0.4:1 and 0.3:1. In certain embodiments the fatty acid oil used in the nanoemulsion were polyunsaturated fatty acids or medium chain triglycerides.

In certain specific embodiments, the nanoemulsion is prepared by applying the PIT method to a mixture of the pre-concentrate and the aqueous phase. The mixture of pre-concentrate and aqueous phase in certain embodiments is spontaneously emulsified such that the oil phase is dispersed among the continuous aqueous phase. The droplet size remains above 200 nm. In certain embodiments the aqueous phase is as defined above. The aqueous phase may include any of those listed herein as possible options for the aqueous phase. As discussed herein, the amount of the aqueous phase in the nanoemulsion is of less importance as compared to the ratio of the oil phase to emulsifier component which includes a surfactant or surfactant blend. Therapeutic agents that are hydrophilic may be added to the aqueous phase while also incorporating the hydrophobic therapeutic agent into the oil phase. The hydrophilic therapeutic agent may include Gentamicin sulfate & bupivacaine HCL in the buffered aqueous phase.

The PIT method used in conjunction with a surfactant or surfactant blend that is suitable for the particular therapeutic agent, produces sub micron nanoemulsion droplets of approximately 120 nm or less using significantly lower surfactant to oil phase ratios as low as 0.3:1. In certain embodiments of the invention the PIT method is modified to add 2-4 additional heating-cooling cycles to the PIT method. These additional cycles reduce the nanoemulsion droplet size to about 100 nm in comparison to the about 200-400 nm typical for cremophore-based nanoemulsion formulations. This in fact allowed for successful sterilization of the produced ISA-247 nanoemulsions using 0.22 μm polycarbonate filter. In certain embodiments of the nanoemulsion, about 50% of the second nanoemulsion droplets have a diameter of less than about 100 nm.

Nanoemulsions were prepared using the low energy PIT method by manipulating the physicochemical properties of the system. This is generally referred to as low-energy emulsification methods. The PIT method changes the spontaneous curvature of the surfactant. For non-ionic surfactants, this can be achieved by changing the temperature of the system, forcing a transition from an oil-in-water (O/W) emulsion at low temperatures to a water-in-oil (W/O) emulsion at higher temperatures (transition phase inversion). During cooling the system crosses a point of zero spontaneous curvature and minimal surface tension, promoting the formation of finely dispersed oil.

In certain embodiments of the invention, the PIT method is used in conjunction with a homogenizer such as the microfluidizer to reduce the droplet sizes of the emulsion. Microfluidization operates at a pressure of greater than 30,000 psi.

In certain embodiments of the present invention, the therapeutic agent, loaded into the oil phase, comprised about 20 wt % of the pre-concentrate, up 30 wt % of the pre-concentrate or up to 40 wt % of the pre-concentrate.

In other embodiments the nanoemulsion is prepared such that the second nanoemulsion has a final therapeutic agent concentration of between about 20 mg/ml and about 60 mg/ml, or between about 30 mg/ml and about 45 mg/ml. All values and sub-ranges within these ranges are contemplated.

Certain aspects of the method of preparing a nanoemulsion formulation further comprise the additional step of sterilizing the second nanoemulsion. The nanoemulsion can be sterilized by passing the nanoemulsion through a polycarobnoate/PTFE 0.2 μm filter.

### Use of Nanoemulsion

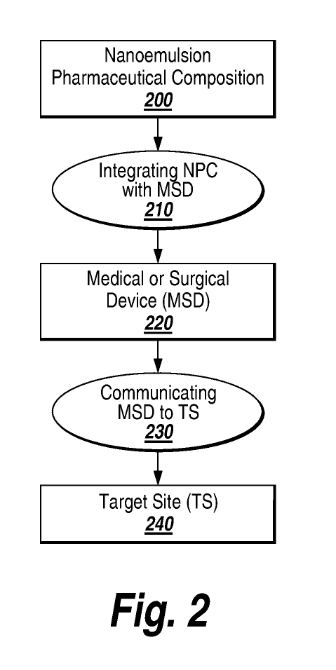

Delivery of the nanoemulsion formulations of the present invention can be achieved by integrating the nanoemulsions of the present invention with a medical or surgical device. Certain aspects of the invention provide a method of administering to a subject a nanoemulsion pharmaceutical composition. The steps, as depicted in the flow chart of FIG. 2, include integrating (210) the nanoemulsion pharmaceutical composition (200) (any of the nanoemulsions provided herein) with a medical or surgical device (220). The term “integrating” is meant to refer to any procedure whereby the nanoemulsion can be placed in contact with a medical or surgical device such that the medical or surgical device may be used to communicate the nanoemulsion to a subject. The steps further include communicating (230) the medical or surgical device to a target site of the subject, such that the nanoemulsion pharmaceutical composition is delivered to the target site (240). Communicating is meant to refer to the process of placing the surgical device in proximate contact with a target site such that the nanoemulsion can be locally or directly transferred from the device to the target site.

In certain embodiments, the invention provides a method of administering the nanoemulsion pharmaceutical composition to a subject in need thereof. Targeting local treatment sites, a therapeutically effective amount of the nanoemulsion pharmaceutical composition as described in any of the above nanoemulsion pharmaceutical composition embodiments is administered via the medical or surgical device. In certain embodiments includes integrating, or applying, a therapeutically effective amount of the nanoemulsion pharmaceutical composition to an irrigation system, a parenteral system, or a topical system and communicating the system to a target site of the subject. In communicating the medical or surgical device to the target site, the device may be intraluminally guided to the target site. In certain embodiments the target site is a body cavity, duct, surface or the like. In certain embodiments the irrigation system includes a micro-porous balloon material. In more preferable embodiments, the irrigation system is a balloon catheter having a porosity of between 0% and 25%. In accordance with aspects of the present invention, the micro-porous balloon is constructed of fluoropolymer material having a microstructure of nodes interconnected by fibrils. The nodes have an internodal distance of about 5 μm to about 60 μm with most embodiments making use of balloons having internodal distances of about 10 μm to about 30 μm. The spaces formed by the internodal distances are oriented to form channels between the nodes extending from an inner surface to an outer surface of the porous delivery device through which a fluid can flow. In other embodiments the irrigation system is adapted for communication to the target site.

As used herein, the term “surface” is used in its broadest sense. In one sense, the term refers to) the outermost boundaries of an organism or inanimate object (e.g., vehicles, buildings, and food processing equipment, etc.) that are capable of being contacted by the compositions of the present invention (e.g., for animals: the skin, hair, and fur, etc., and for plants: the leaves, stems, flowering parts, and fruiting bodies, etc.). In another sense, the term also refers to the inner membranes and surfaces of animals and plants (e.g., for animals: the digestive tract, vascular tissues, and the like, and for plants: the vascular tissues, etc.) capable of being contacted by compositions by any of a number of transdermal delivery routes (e.g., injection, ingestion, transdermal delivery, inhalation, and the like).

As used herein, the term “topical,” or “topically” refers to application of the compositions of the present invention to the surface of the skin and mucosal cells and tissues (e.g., alveolar, buccal, lingual masticatory, or nasal mucosa, and other tissues and cells which line hollow organs or body cavities).

Though any suitable medical or surgical devices are contemplated, preferred medical or surgical devices include an IV bag or an irrigation system. In certain embodiments, the medical or surgical device is an irrigation system. Irrigation systems include a balloon catheter. In certain embodiments, the irrigation system is a balloon catheter having a perforated or micro-porous portion. The perforation or micro-porosity of the portion may be due to the natural characteristics of the material used to form a balloon portion of the catheter, or the porosity may be a manufactured characteristic. The micro-porous portion of the balloon catheter is a balloon having a porosity of greater than 0 and less than about 25% and an internodal distance of about 5 μm to about 60 μm, or about 10 μm to about 30 μm. In all cases, the irrigation systems used in the methods provided herein, are adapted for communication to a target site. The target site as used herein refers to any surface, cavity, duct, pathway, or other distal part of a subject's body where the irrigation system may be communicated. Target sites include, for example, arteries, vaginal, oral or anal cavity. Accordingly, the medical device, or irrigation system, is adapted for parenteral, mucosal, topical or ocular administration to the target site.

The invention, in part, is directed to integrating the nanoemulsion pharmaceutical composition provided herein, with the medical or surgical device such that the device can be used to locally deliver the therapeutic agent that has been emulsified in the nanoemulsion. Catheters are contemplated as suitable medical or surgical devices to be integrated with the nanoemulsion pharmaceutical composition.

The present invention contemplates methods where a balloon catheter or other irrigation system, is integrated with the nanoemulsion and is delivered to an artery, such as where a surgery is being performed, and the nanoemulsion is allowed to pass through a micro-porous portion of the balloon catheter. The droplet size of the nanoemulsion enables a controllable flow rate such that the emulsified therapeutic agent can be locally delivered to the target site within the artery, for example. For example, the nanoemulsion pharmaceutical composition is integrated with a micro-porous portion of the balloon catheter via a lumen attached at a distal end to the balloon portion such that the nanoemulsion pharmaceutical composition traverses the pores of the balloon portion with a flow rate that is sufficient to effectively treat the target site. A flow rate is effective if the therapeutic agent is allowed to penetrate the neighboring tissue proximate the balloon portion.

A balloon catheter is a type of soft catheter with an inflatable balloon at its tip which is used during a catheterization procedure to enlarge a narrow opening or passage within the body. The deflated balloon catheter is positioned, then inflated to perform the necessary procedure, and deflated again in order to be removed. Examples of suitable balloon catheters for administration of the nanoemulsion, includes CLEARWAY® (micro-porous balloon catheter).

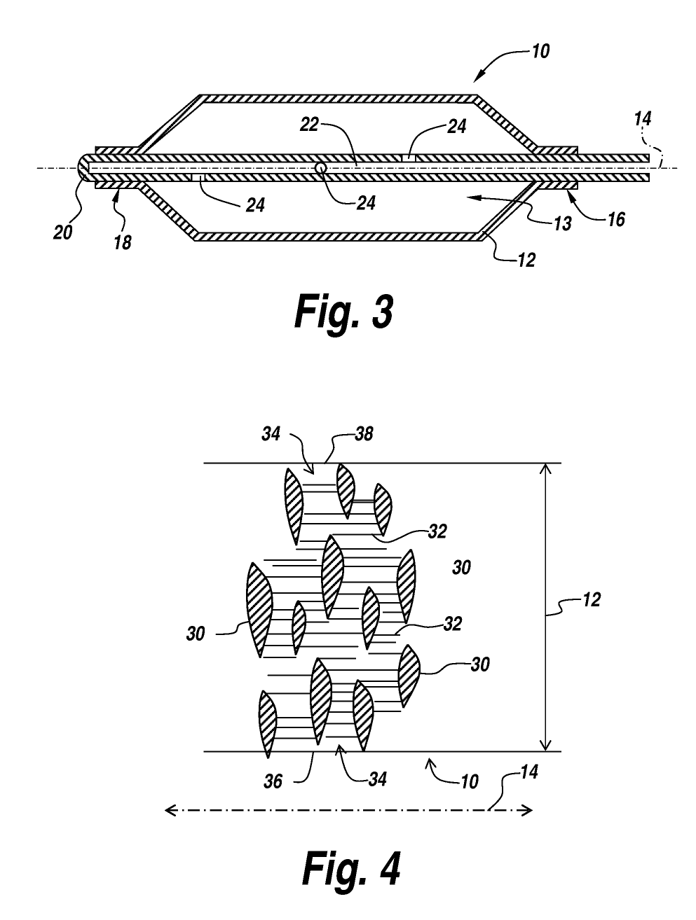

Referring specifically to FIG. 3, an example micro-porous balloon 10 is shown having a micro-porous body 12. The micro-porous balloon 10 can have a number of different applications and uses. The micro-porous balloon 10 is preferably generally tubular in shape when expanded, although other cross-sections, such as rectangular, oval, elliptical, or polygonal, can be also be utilized with this inflation coating deployment system. The cross-section of the micro-porous body 12 is preferably continuous and uniform along the length of the body. However, in alternative embodiments, the cross-section can vary in size and/or shape along the length of the body. The micro-porous body 12 has a central lumen 13 extending along a longitudinal axis 14 between a first end 16 and second end 18.

An inflation mechanism in the form of an elongated hollow, porous tube 20 is shown positioned within the central lumen 13 to provide a radial deployment or expansion force to the micro-porous body 12. The radial deployment force effects radial expansion of the micro-porous body 12 from the first configuration to the second increased diameter configuration illustrated in FIG. 3. The first end 16 and the second end 18 are connected in sealing relationship to the outer surface of the hollow, porous tube 20. The first and second ends 16 and 18 can be thermally bonded, bonded by means of an adhesive, or attached by other mechanical sealing means suitable for inhibiting fluid leakage from the first and second ends 16 and 18 between the walls of the micro-porous body 12 and the porous tube 20.

FIG. 4 is a schematic representation of the microstructure of the micro-porous body 12 of the radially expandable shape form device 10. For purposes of description, the microstructure of the wall of the micro-porous body 12 has been exaggerated. Accordingly, while the dimensions of the microstructure are enlarged, the general character of the illustrated microstructure is representative of the porous node and fibril microstructure prevailing within the wall of the micro-porous body 12.

The microstructure of the wall of the micro-porous body 12 is characterized by nodes 30 interconnected by fibrils or filaments 32. The nodes 30 are generally oriented perpendicular to the longitudinal axis 14 of the micro-porous body 12. This microstructure of nodes 30 interconnected by fibrils 32 provides a porous microstructure having microfibrillar spaces which define channels or through-pores 34 extending entirely from the inner wall surface 36 to the outer wall surface 38 of the micro-porous body 12. The through-pores 34 are perpendicularly oriented (relative to the longitudinal axis 14), internodal spaces that traverse from the inner wall surface 36 to the outer wall surface 38. The size and geometry of the through-pores 34 can be altered through the chemical preparation, formulation, extrusion and or material stretching and sintering process, as described in detail in U.S. Pat. No. 6,955,661, filed on Oct. 1, 1999, which is incorporated herein by reference. The size and geometry of the through-pores 34 can also be altered to yield a radially expandable microstructure that consists of nodes and fibrils that can result in shaped form. The shaped form can be made selectively impermeable, semi-impermeable, or permeable by various inflation liquid viscosities, various inflation pressures or any combinations of liquid inflation viscosities and inflation pressures.

Without extensive discussion, it will be assumed for purposes of example, that the catheter body employed for the present invention involves a simple single or double lumen, one or two tube, catheter construction with some auxiliary mechanism, such as a wire or cable for affecting a push-pull motion at the tip of the catheter. A number of mechanisms for affecting such capabilities are known in the art and may be substituted for the described elements wherever appropriate.

Delivery of certain therapeutic agents that are poorly water-soluble and consequently have low bioavailability are improved by the present invention. It has been surprisingly found that the solubility and bioavailability of certain poorly water-soluble therapeutic agents is improved by incorporating said therapeutic agents into a nanoemulsion formulation and using a medical or surgical device to directly deliver the therapeutic agent to a target site of a subject's body.

The bioavailability of hydrophobic therapeutic agents is improved by the emulsions provided herein. In certain exemplary embodiments of the present invention it has been surprisingly found that a nanoemulsion pharmaceutical composition prepared, by the Phase Inversion Temperature (PIT) method, from a pre-concentrate and an aqueous phase contains nanoemulsion droplets small enough for delivery to a subject via a medical delivery device having a micro-porous member used to transport and deliver the nanoemulsion to a target site of the subject. The nanoemulsion droplets contain an emulsified therapeutic agent. These nanoemulsion formulations have been found to be excellent vehicles to solubilize lipophilic drugs and significantly improve bioavailability. Smaller nanoemulsion droplet size dramatically increases the surface volume ratio and therefore significantly enhances the bioavailability and efficacy of the hydrophobic therapeutic agent. There are also other factors that affect delivery such as the surface rheological properties of droplets. Surface rheological properties which are affected by surfactant directly influence the diffusion of the emulsified therapeutic agent. Due to the physico-chemical properties and structures of surfactants, it can increase permeation and facilitate delivery of the therapeutic agent. The nanoemulsion pharmaceutical compositions of the invention herein can be administered systemically, parenterally, topically or orally.

Nanoemulsions of certain embodiments of the present invention have at least one dimension less than 100 nm. Due to their small droplet sizes, nanoemulsions may appear transparent or translucent. The Brownian motion associated with nanoemulsions prevents their sedimentation or creaming and hence the nanoemulsions offer increased stability. For example, nanoemulsions are metastable and can be diluted with water without changing the droplet distribution.

The nanoemulsion formulations can be used as excellent vehicles to solubilize lipophilic drugs and significantly improved bioavailability. Smaller droplet size dramatically increases the surface volume ratio and therefore significantly enhances the bioavailability and efficacy. The physicochemical properties and structures of surfactants can increase permeation and facilitate drug delivery.

### EXAMPLES

The following examples are for illustrative purposes and are not intended to be exhaustive of the possible embodiments of the invention herein provided.

### Example 1

The stability of the nanoemulsion is demonstrated in this example wherein the emulsifying component is Tween 80 and the various oil phases consist of fish oil, Vitamin E or a mixture of Vitamine E and fish oil. The emulsion was neither microfluidized nor subjected to the PIT or modified PIT method. The results, demonstrated in table 1, show the large particle sizes and that the emulsions are unstable as determined by a loss of desired physical properties after 1 hour.

TABLE 1

| Emulsion | | | |
|---|---|---|---|
| formulation | | Zeta | |
| No | Droplet | potential | Physical appearance |
| Microfluidization | size (nm) | (meV) | (1 hr-Stability, RT) |
| 100% fish oil | 1740 | 2.49 | Non-homogenous oily, |
| | | | creamy opaque emulsion |
| 100% Vit E | N/A | −15.7 | Non-homogenous oily, |
| | (>6000 nm) | | yellow creamy opaque |
| | | | emulsion |
| 70% F.O:30% Vit E | N/A | −18 | Non-homogenous oily, |
| | (>6000 nm) | | creamy opaque emulsion |
| 30% F.O:70% Vit E | N/A | −20.7 | Non-homogenous oily, |
| | (>6000 nm) | | creamy opaque emulsion |

### Example 2

Example 2 demonstrates the stability of nanoemulsions emulsified by Tween 80 where the nanoemulsion is prepared by a microfluidizer. While improved physical stability is observed in Table 2, relative to example 1, the nanoemulsion remains relatively unstable as compared to emulsions wherein a PIT method is also employed.

TABLE 2

| Nanoemulsion formu- | | | |
|---|---|---|---|
| lation (Processed via | | Zeta | |
| the M-110EH-30 Micro- | Droplet | potential | Physical appearance |
| fluidizer Processor) | size (nm) | (meV) | (1 hr-stability, RT) |
| 100% fish oil | 97.7 | −18.4 | Homogenous |
| | | | milky liquid |
| 100% Vit E | 131.0 | −17.0 | Homogenous |
| | | | yellowish creamy |
| | | | opaque liquid |
| 70% F.O:30% Vit E | 98.2 | −13.8 | Homogenous |
| | | | milky liquid |
| 30% F.O:70% Vit E | 91.7 | −21.6 | Homogenous |
| | | | yellowish |
| | | | white creamy |
| | | | opaque liquid |

### Example 3

In this second example, Cremaphor EL was used to emulsify the emulsion. A combination of the microfluidizer and the PIT method was used to reduce the nanoemulsion droplet size. The stability of the nanoemulsion was demonstrated by the physical appearance of the emulsion, as presented in Table 3. Nanoemulsions having a mixture of fish oil and Vitamin E produced the most stable nanoemulsion formulations having a clear and homogenous appearance. Additionally, using the combination of the microfluidizer and PIT combination methods with using a combination of vitamin E and fish oil in the oil phase of the emulsion a much smaller particle size was achieved.

TABLE 3

| Nanoemulsion formu- | | | |
|---|---|---|---|
| lation (Processed via | | Zeta | |
| the M-110EH-30 Micro- | Droplet | potential | Physical appearance |
| fluidizer Processor) | size (nm) | (meV) | (Stability after 1 hr) |
| 100% fish oil | 115 | −28.7 | Homogenous |
| | | | milky liquid |
| 100% Vit E | 70.5 | −12.9 | Homogenous |
| | | | yellowish liquid |
| 70% F.O:30% Vit E | 23.8 | −12.4 | Homogenous |
| | | | clear yellowish |
| | | | liquid |
| 30% F.O:70% Vit E | 31.9 | −14.0 | Homogenous |
| | | | clear yellowish |
| | | | liquid |

### Example 4

As a fourth example, the following nanoemulsion formulation for systemic delivery of an emulsified therapeutic agent provides a nanoemulsion using various combinations of fish oil and Vitamin E to achieve submicron nanoemulsion droplets. The oil phase was 15% of the total nanoemulsion formulation. The aqueous phase included 78% of the total nanoemulsion formulation and the emulsifier component included 7% of the total nanoemulsion formulation. The flow rate of this nanoemulsion formulation through the CLEARWAY® device was examined with the results shown in Table 4. These results show the applicability of using these formulations to deliver therapeutic compounds through the CLEARWAY® device in local drug delivery applications.

TABLE 4

| Nano-emulsion formu- | | | |
|---|---|---|---|
| lation (classified | Droplet size | | Flow rate through |
| based on oil-phase | (nm)& zeta | Vis- | CLEARWAY ® |
| composition) | potential(mV) | cosity | (mL/min, at 4 ATM) |
| 100 Fish oil | 115/−28 | 2.26 | 2.3 |
| 100 Vitamin E | 70.5/−12.9 | 2.73 | ~3 |
| Fish oil:Vit. E | 23.8/−12.4 | 2.45 | 1.8-2 |
| 70:30% wt | | | |
| Fish oil:Vit. E | 31.9/−14 | 2.51 | 2.9-3 |
| 30:70% wt | | | |

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the invention, and exclusive use of all modifications that come within the scope of the provided invention is reserved.

What is claimed is:

1. A pre-concentrate, comprising:
an oil phase;
a therapeutic agent; and
an emulsifier component;
wherein the ratio of emulsifier component to oil phase is about 1:1 or less.

2. The pre-concentrate of claim 1, wherein the ratio of the emulsifier component to the oil phase is about 0.4:1 or less.

3. The pre-concentrate of claim 1, wherein the ratio of the emulsifier component to the oil phase is about 0.3:1 or less.

4. The pre-concentrate of claim 1, wherein the oil phase comprises one or more fatty acid oils.

5. The pre-concentrate of claim 4, wherein the one or more fatty acid oils include a polyunsaturated fatty acid.

6. The pre-concentrate of claim 4, wherein the one or more fatty acid oils include a medium chain triglyceride (MCT).

7. The pre-concentrate of claim 5, wherein the polyunsaturated fatty acid includes an omega-3 fatty acid.

8. The pre-concentrate of claim 5, wherein the polyunsaturated fatty acid is eicosapentaenoic acid, salts of eicosapentaenoic acid, docosahexaenoic acid, salts of docosahexaenoic acid, triglycerides of eicosapentaenoic acid, tryglycerides of docosahexaenoic acid, ethyl esters of eicosapentaenoic acid, or ethyl esters of docosahexaenoic acid.

9. The pre-concentrate of claim 7, wherein the polyunsaturated fatty acid includes at least one fish oil.

10. The pre-concentrate of claim 1, wherein the oil phase comprises a mixture of at least one fish oil and vitamin E.

11. The pre-concentrate of claim 10, wherein the ratio of fish oil to vitamin E is between about 3:7 and about 9:1.

12. The pre-concentrate of claim 11, wherein the ratio of fish oil to vitamin E is 7:3.

13. The pre-concentrate of claim 1, wherein the therapeutic agent is hydrophobic and is substantially miscible in the oil phase.

14. The pre-concentrate of claim 1, wherein the therapeutic agent is up to approximately 50 wt % of the oil phase.

15. The pre-concentrate of claim 1, wherein the therapeutic agent is one or more of an anti-inflammatory agent, analgesic, anti-allergenic, anti-fungal, anti-arrhythmic agent, antibiotic, anticoagulant, antidepressant, antidiabetic agent, anti-epilepsi agent, anti-hypertensive agent, anti-gout agent, anti-malarial, anti-migraine agent, antimuscarinic agent, antineoplastic agent, anti-protozoal agent, anxiolytic, thyroid, anti-thyroid, antiviral, anoretic, bisphosphonate, cardiac inotropic agent, cardiovascular agent, corticosteroid, diuretic, dopaminergic agent, gastrointestinal agent, hemostatic, histamine receptor antagonist, hypnotic, immunosuppressant, kidney protective agent, lipid regulating agent, muscle relaxant, neuroleptic, neurotropic agent, opioid agonist, and antagonist, parsympathomimetic, protease inhibitor, prostaglandin, sedative, sex hormone, stimulant, sympathomimetic vasodilator, and xanthan, or mixtures thereof.

16. The pre-concentrate of claim 15, wherein the anti-inflammatory agent is a cyclosporine derivative.

17. The pre-concentrate of claim 16, wherein the therapeutic agent is ISA-247.

18. The pre-concentrate of claim 1, wherein the therapeutic agents is selected from the group consisting of Amphotericin B, TAFA93, SAR943, ISA247, rapamycin, cyclosporine, cyclosporine A, other cyclosporine derivatives and rapamycin derivatives.

19. The pre-concentrate of claim 1, wherein the emulsifier component comprises one or more surfactants.

20. The pre-concentrate of claim 19, wherein the one or more surfactants is selected from the group consisting of Vitamin E TPGS, lecithin, SolutolHS-15, polysorbate 80, and cremophore EL.

21. The pre-concentrate of claim 20, wherein the one or more surfactants are cremophore EL.

22. A nanoemulsion pharmaceutical composition, comprising:
a pre-concentrate, comprising:
an oil phase;
a therapeutic agent; and
an emulsifier component;
wherein the ratio of emulsifier component to oil phase is about 1:1 or less; and
an aqueous medium;
wherein the emulsifier component is less than about 10 wt % of the nanoemulsion pharmaceutical composition.

23. The nanoemulsion pharmaceutical composition of claim 22, wherein the oil phase is loaded up to 50% with the therapeutic agent.

24. The nanoemulsion pharmaceutical composition of claim 22, wherein the therapeutic agent is about 5 wt % of the nanoemulsion pharmaceutical composition.

25. The nanoemulsion pharmaceutical composition of claim 22, wherein the therapeutic agent is up to approximately 30 wt % of the oil phase.

26. The nanoemulsion pharmaceutical composition of claim 22, wherein the therapeutic agent is ISA-247.

27. The nanoemulsion pharmaceutical composition of claim 22, wherein the oil phase is between about 10 wt % and about 15 wt % of the nanoemulsion pharmaceutical composition.

28. The nanoemulsion pharmaceutical composition of claim 22, wherein the aqueous medium includes water, glycofurol, ethyl acetate, propylene glycol, ethanol, lower alkanols, or mixtures thereof.

29. The nanoemulsion pharmaceutical composition of claim 22, wherein the aqueous medium is between about 75 wt % and about 85 wt % of the nanoemulsion pharmaceutical composition.

30. The nanoemulsion pharmaceutical composition of claim 22, wherein the emulsifier component is between about 5 wt % and about 15 wt % of the nanoemulsion pharmaceutical composition.

31. The nanoemulsion pharmaceutical composition of claim 22, wherein the emulsifier component is less than about 5 wt % of the nanoemulsion pharmaceutical composition.

32. The nanoemulsion pharmaceutical composition of claim 22, wherein the nanoemulsion pharmaceutical composition has a final therapeutic agent concentration of between about 20 mg/ml and about 60 mg/ml.

33. The nanoemulsion pharmaceutical composition of claim 32, wherein a final concentration of the therapeutic agent is between about 30 mg/ml and about 45 mg/ml.

34. The nanoemulsion pharmaceutical composition of claim 22, wherein the nanoemulsion contains droplets having a diameter range between about 30 and about 200 nanometers.

35. The nanoemulsion pharmaceutical composition of claim 34, wherein the droplets have a diameter of less than about 120 nm.

36. The nanoemulsion pharmaceutical composition of claim 22, wherein the nanoemulsion pharmaceutical composition is adapted for communication from an irrigation system to a body cavity.

37. The nanoemulsion pharmaceutical composition of claim 22, wherein the nanoemulsion has a zeta-potential range from about 0 to about −40 meV.

38. The nanoemulsion pharmaceutical composition of claim 22, wherein the nanoemulsion has a viscosity range from about 2.0 to about 3.0.

39. A nanoemulsion, comprising:
a nanoemulsion pre-concentrate which comprises a mixture of fish oil and vitamin E, a therapeutic agent, and cremophore EL; and
water;
wherein, the nanoemulsion has oil-in-water emulsion droplets and at least 50% of the emulsion droplets have a diameter of less than 100 nm;
wherein the cremophore EL comprises less than 5 wt % of the nanoemulsion; and
wherein the ratio of cremophore EL to the mixture of fish oil and Vitamin E is about 0.3:1 or less.

40. The nanoemulsion of claim 39, wherein the therapeutic agent is ISA-247.

41. A method of preparing a nanoemulsion formulation, comprising:
preparing the pre-concentrate of claim 1 to form a first mixture;
adding the first mixture to an aqueous phase to form a second mixture;
applying a microfluidizer to the second mixture to form a first nanoemulsion; and
applying at least one PIT cycle to the first nanoemulsion to form a second nanoemulsion wherein at least 70% of nanoemulsion droplets forming the second nanoemulsion have a diameter of about 200 nm or less.

42. The method of claim 41, wherein 2-4 additional PIT cycles are applied to the first nanoemulsion.

43. The method of claim 41, wherein the therapeutic agent is a cyclosporine derivative.

44. The method of claim 41, wherein the therapeutic agent is at least about 20 wt % of the pre-concentrate.

45. The method of claim 41, wherein the therapeutic agent is at least about 30 wt % of the pre-concentrate.

46. The method of claim 41, wherein the second nanoemulsion pharmaceutical composition has a final therapeutic agent concentration of between about 20 mg/ml and about 60 mg/ml.

47. The method of claim 46, wherein a final concentration of the therapeutic agent is between about 30 mg/ml and about 45 mg/ml.

48. The method of claim 41, wherein about 50% of the second nanoemulsion droplets have a diameter of less than about 100 nm.

49. The method of claim 41, further comprising the step of sterilizing the second nanoemulsion by passing the second nanoemulsion through a polycarbonate/PTFE 0.2 μm filter.

50. The method of claim 41, wherein the oil phase comprises a polyunsaturated fatty acid.

51. The method of claim 50, wherein the polyunsaturated fatty acid is selected from the group consisting of eicosapentaenoic acid, salts of eicosapentaenoic acid, docosahexaenoic acid, salts of docosahexaenoic acid, triglycerides of eicosapentaenoic acid, tryglycerides of docosahexaenoic acid, ethyl esters of eicosapentaenoic acid, and/or ethyl esters of docosahexaenoic acid.

52. The method of claim 50, wherein the polyunsaturated fatty acid is an omega-3 fatty acid.

53. The method of claim 52, wherein the omega-3 fatty acid is a fish oil or a mixture of different fish oils.

54. The method of claim 41, wherein the oil phase comprises a mixture of fish oil and vitamin E.

55. The method of claim 54, wherein the ratio of fish oil to vitamin E is between about 3:7 and about 1:1.

56. The method of claim 41, wherein the therapeutic agent is substantially miscible in the fatty acid oil component.

57. The method of claim 41, wherein the ratio of the emulsifier component to the oil phase is about 0.3:1 or less.

58. The method of claim 41, wherein the second nanoemulsion has a zeta-potential ranging from about 0 to about −40 meV.

59. The method of claim 41, wherein the second nanoemulsion has a viscosity ranging from about 2.0 to about 3.

60. A method of administering to a subject a nanoemulsion pharmaceutical composition, comprising:
integrating a nanoemulsion pharmaceutical composition comprising a pre-concentrate according to claim 1 and an aqueous medium wherein the emulsifier component is less than about 10 wt % of the nanoemulsion pharmaceutical composition with a medical or surgical device; and
communicating the medical or surgical device to a target site of the subject such that the nanoemulsion pharmaceutical composition is delivered to the target site.

61. The method of claim 60, wherein the medical or surgical device is an irrigation system.

62. The method of claim 61, wherein the medical or surgical device is an IV bag.

63. The method of claim 61, wherein the irrigation system is a balloon catheter having a perforated or micro-porous portion.

64. The method of claim 63, wherein the micro-porous portion of the balloon catheter is a balloon having a porosity of greater than 0 and less than about 25%.

65. The method of claim 63, wherein the nanoemulsion pharmaceutical composition is integrated with a micro-porous balloon portion of the balloon catheter via a lumen attached at a distal end to the balloon portion such that the nanoemulsion pharmaceutical composition traverses pores of the balloon portion with a flow rate sufficient to effectively treat the target site.

66. The method of claim 61, wherein the irrigation system is adapted for communication to the target site.

67. The method of claim 60, wherein the target site is any body cavity, duct or surface of the subject.

68. The method of claim 60, wherein the medical or surgical device is adapted for parenteral, mucosal, topical or ocular administration to the target site.

69. A nanoemulsion delivery kit, comprising:
a pre-concentrate, comprising:
an oil phase;
a therapeutic agent; and
an emulsifier component;
wherein the ratio of emulsifier component to oil phase is about 1:1 or less;
an aqueous medium; and
a medical or surgical device configured as a delivery mechanism for delivery of a nanoemulsion formed using the pre-concentrate and the aqueous medium.
